(19) United States
(12) Patent Application Publication
(10) Pub. No.: US 2011/0159066 A1
(43) Pub. Date: Jun. 30, 2011

(54) Methods For Treating Parkinson's Disease

(76) Inventors: Terri B. Sebree, Gladwyne, PA (US); Steven J. Siegel, Berwyn, PA (US)

(21) Appl. No.: 12/938,136

(22) Filed: Nov. 2, 2010

Related U.S. Application Data
(60) Provisional application No. 61/257,418, filed on Nov. 2, 2009.
(60) Provisional application No. 61/376,522, filed on Aug. 24, 2010.

(51) Int. Cl. A61K 31/4985; A61K 31/404; A61F 2/00; A61K 31/198; A61K 31/437; A61K 31/428; A61K 31/496; A61P 25/16

(52) U.S. Cl. 424/422; 514/418; 514/567; 514/250; 514/288; 514/367; 514/252.2

(57) ABSTRACT

Methods for restoring normal patterns of activity in a subject suffering from Parkinson's Disease are disclosed that include administering an effective steady state concentration of a dopamine modulating compound continuously for a prolonged period of time such that normal patterns of activity are substantially restored in the subject.

### RELATED APPLICATIONS

This application is related and claims priority to U.S. Provisional Application Ser. No. 61/257,418, filed Nov. 2, 2009 and U.S. Provisional Application Ser. No. 61/376,522, filed Aug. 24, 2010. The entire contents of these applications are hereby incorporated by this reference.

### BACKGROUND OF THE INVENTION

Parkinson's disease is a progressive degenerative disease of the central nervous system. The risk of developing Parkinson's disease increases with age, and afflicted individuals are usually adults over 40. Parkinson's disease occurs in all parts of the world, and affects more than one million individuals in the United States alone.

While the primary cause of Parkinson's disease is not known, it is characterized by degeneration of dopaminergic neurons of the substantia nigra. The substantia nigra is a portion of the lower brain, or brain stem, that helps control voluntary movements. The shortage of dopamine in the brain caused by the loss of these neurons is believed to cause the observable disease symptoms.

The symptoms of Parkinson's disease vary from patient to patient. The most common symptom is a paucity of movement, e.g., rigidity characterized by an increased stiffness of voluntary skeletal muscles. Additional symptoms include resting tremor, bradykinesia (slowness of movement), poor balance, and walking problems. Common secondary symptoms include depression, sleep disturbance, dizziness, stooped posture, dementia, and problems with speech, breathing, and swallowing. The symptoms become progressively worse and ultimately result in death.

Surgical treatments available for Parkinson's disease include pallidotomy, brain tissue transplants, and deep brain stimulation. Such treatments are highly invasive procedures accompanied by the usual risks of brain surgery, including stroke, partial vision loss, speech and swallowing difficulties, and confusion.

A variety of chemotherapeutic treatments for Parkinson's disease are also available, including levodopa, a dopamine precursor. While levodopa administration can result in a dramatic improvement in symptoms, patients can experience serious side-effects, including nausea and vomiting. Concurrent carbidopa administration with levodopa is a significant improvement, with the addition of carbidopa inhibiting levodopa metabolism in the gut, liver and other tissues, thereby allowing more levodopa to reach the brain. Additional therapeutic approaches include the use of dopamine agonists such as ropinirole, pergolide and apomorphine.

All current treatments for Parkinson's Disease (PD) require dosing of one or more times per day. This pattern of administration leads to a cycle of remission and reemergence of symptoms with periods of relative remission interspersed between periods of bradykinesia and dyskinesia. However, several studies suggest that continuous delivery of anti-Parkinsonian medications, including dopamine agonists to could alleviate transitions between these so called “on” and “off” times. Previous efforts have capitalized upon this approach to deliver L-dopa or apomorphine through infusion pumps with good therapeutic results including reduction in both bradykinesia and dyskinesia. However, intra-intestinal or subcutaneous pumps are often difficult to tolerate.

### SUMMARY OF THE INVENTION

Accordingly, in some embodiments, the present invention provides a method for restoring normal patterns of activity in a subject suffering from Parkinson's Disease. The method includes administering an effective steady state concentration of a dopamine modulating compound continuously for a prolonged period of time such that normal patterns of activity are substantially restored in the subject.

In some embodiments, the present invention provides a method for increasing on time in a subject suffering from Parkinson's Disease. The method includes administering an effective steady state concentration of a dopamine modulating compound, alone or combination with another therapy, continuously for a prolonged period of time, such that on time is increased in the subject.

In some embodiments, off time is reduced in the subject. In some embodiments, the severity of off time symptoms are reduced in the subject. In some embodiments, the frequency of off time symptoms are reduced in the subject. In some embodiments, the incidence of motor response complications are reduced in the subject. In some embodiments, there are no significant and/or prolonged periods of hyperkinetic activity in the subject. In some embodiments, there are no periods of hyperkinetic activity in the subject. In some embodiments, the subject experiences a period of on time immediately after waking up from sleep. In some embodiments, the subject does not experience paralysis.

In some embodiments, the dopamine modulating compound is administered without the side effects associated with administration by pump infusion.

In some embodiments, sustained efficacy is achieved in the subject for greater than 30 days.

In some embodiments, the delivery dose required to achieve the same pharmacokinetic profile as an approved orally administered dose is 1/9 or 1/18 that of the approved orally administered dose.

In some embodiments, the dopamine modulating compound is delivered via an implant. In some embodiments, the dopamine modulating compound is delivered via an implant which comprises a core comprising a dopamine modulating compound and a first biodegradable polymer; and a sheath comprising a second biodegradable polymer. In some embodiments, the dopamine modulating compound is delivered via a depot.

In some embodiments, the dopamine modulating compound is co-administered with another therapy selected from dopamine metabolic inhibitors, monoamine oxidase inhibitors, dopaminergics, dopamine agonists or adenosine receptor antagonists. In some embodiments, the amount of the co-administered therapy administered is significantly decreased over time. In some embodiments, the side effects corresponding to the co-administered therapy are significantly reduced. In some embodiments, the co-administered therapy is a dopaminergic, e.g., L-Dopa.

In some embodiments, the dopamine modulating compound is a 4-alkylamino-2(3H)-indolone compound. In some embodiments, the dopamine modulating compound is selected from bromocriptine, pergolide, pramipexole, ropinirole, piribedil, cabergoline, and lisuride. In some embodiments, the dopamine modulating compound is ropinirole.

In some embodiments, the present invention provides a method for the treatment of Parkinson's Disease in a patient in need thereof. The method includes administering a continuous and prolonged delivery of ropinirole via an implant, in combination with L-Dopa, wherein on time is increased and off time is decreased.

In some embodiments, the subject is capable of normal activity during sleep. In some embodiments, the patient is capable of normal movement continuously.

In some embodiments, the dopamine modulating compound is administered such that normal patterns of activity are substantially restored in the subject immediately after waking up from sleep. In some embodiments, the dopamine modulating compound is administered such that normal patterns of activity are substantially restored in the subject for at least 18 hours per day.

In some embodiments, the Parkinson's Disease is mild to moderate Parkinson's Disease.

In some aspects, the invention provides methods for treating a subject for a dopamine associated state, comprising administering to said subject an implant to comprised of one or more biodegradable implant sections of any one of the preceding claims, wherein said implant releases an effective amount of a dopamine modulating compound over a treatment period, such that said subject is treated for said dopamine associated state.

In some embodiments, dopamine associated state is Parkinson's disease, attention deficit disorder (ADD), attention deficit hyperactivity disorder (ADHD), autism, pervasive development disorder (PDD), Asberger's syndrome, toxin-induced Parkinsonism, disease-induced Parkinsonism, erectile dysfunction, restless leg syndrome, or hyperprolactinemia.

In some embodiments, the amount of said dopamine modulating compound released varies less than about ±20% or less than about ±10% during said treatment period.

In some embodiments, the treatment period is from about 40 days to about 80 days.

In some embodiments, the amount of the dopamine modulating compound released is such that side effects are reduced.

### BRIEF DESCRIPTION OF THE DRAWINGS

FIG. 1 is a graph showing the mean pharmacokinetic profile obtained with exemplary implant (NP201) in a primate model of Parkinson's Disease;

FIG. 2 is a graph showing the mean pharmacokinetic results comparing NP201 and oral ropinirole;

FIG. 3 is a graph showing the Clinical Rating Scale (CRS) change from baseline standardized rank repeated measures analysis;

FIG. 4 is a graph showing pre-MPTP baseline activity (Wed-Fri and Sat-Sun);

FIG. 5 is a graph showing activity data for days 10-42 (Wed-Fri);

FIG. 6 is a graph showing activity data for days 10-42 (Wed-Fri) and pre-MPTP baseline (Wed-Fri);

FIG. 7 is a graph showing activity data for days 10-42 (Sat-Sun);

FIG. 8 is a graph showing activity data for days 10-42 (Sat-Sun) and pre-MPTP baseline (Sat-Sun);

FIG. 9 is a graph showing placebo activity data for days 10-42 (Wed-Fri and Sat-Sun);

FIG. 10 is a graph showing oral activity data for days 10-42 (Wed-Fri and Sat-Sun);

FIG. 11 is a graph showing NP201 activity data for days 10-42 (Wed-Fri and Sat-Sun);

FIG. 12 is a graph showing total activity within a 24 hour period (Wed, Thurs, Fri); and

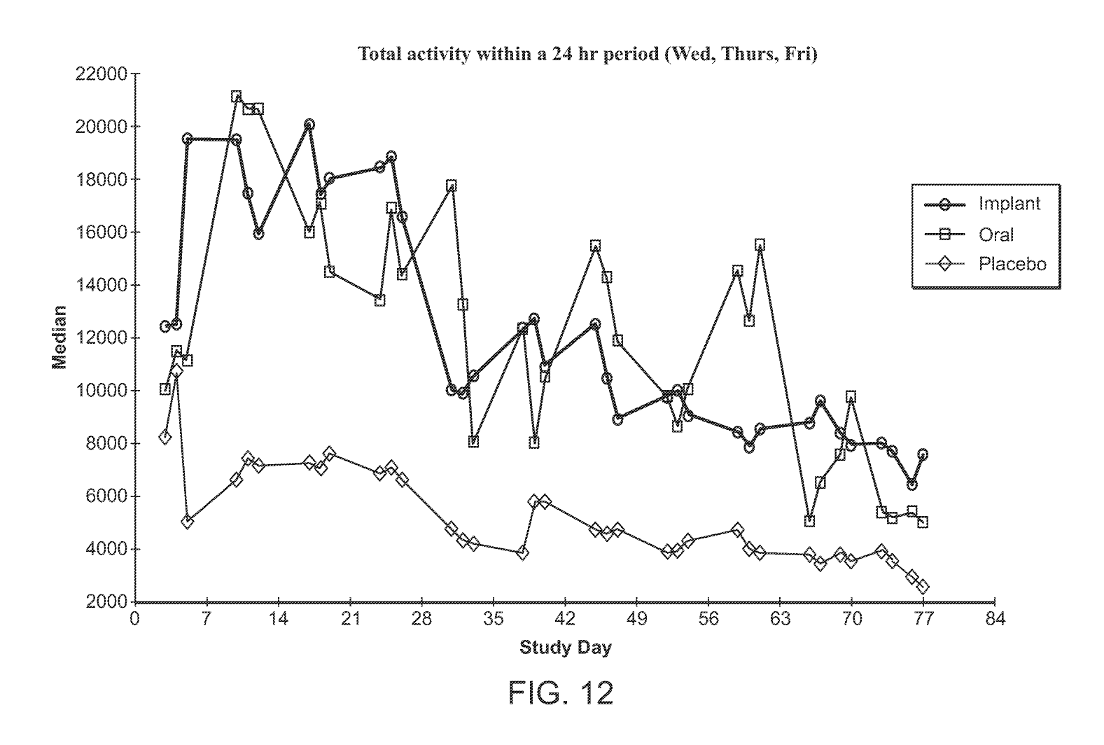

FIG. 13 depicts an exemplary biodegradable sustained-release ropinirole implant (NP201).

### DETAILED DESCRIPTION OF THE INVENTION

The present invention is based, at least in part, on the discovery that long term sustained delivery of a dopamine modulating compound, e.g., via an implant as described herein, allows a subject to maintain patterns of normal activity. Dopamine associated states, such as Parkinson's Disease, may be characterized by bradykinesia and/or dyskinesia. Without wishing to be bound by any particular theory, it is believed that sustained delivery of a dopamine modulating compound (e.g., for at least 15, 30, 45, 60 days or more) allows for an amount of compound in the blood which minimizes these symptoms. For example, oral dosing using ropinerole often leads to bradykinesia in the morning. This makes simple tasks, such as using the bathroom or taking medications, very difficult. The methods described herein can allow for normal patterns of activity, not only in the morning, but also throughout the day.

Accordingly, in some embodiments, the present invention provides methods for restoring normal patterns of activity in a subject suffering from a dopamine associated state, e.g., Parkinson's Disease. Such methods include administering an effective steady state concentration of a dopamine modulating compound continuously for a prolonged period of time such that normal patterns of activity are substantially restored in the subject. As used herein, the term “prolonged period of time” refers to a period of at least about 15 days, at least about 30 days, at least about 45 days, at least about 60 days, at least about 75 days, at least about 90 days or more. As used herein, the phrase “normal patterns of activity” refer to patterns of activity which include no or little bradykinesia or dyskinesia. In some embodiments, “normal patterns of activity” include patterns of activity in a subject with a Clinical Rating Scale of less than about 7. A discussion of the Clinical Rating Scale can be found in the Examples.

In other embodiments, the present invention provides a method for increasing on time in a subject suffering from a dopamine associated state, e.g., Parkinson's Disease. Such methods include administering an effective steady state concentration of a dopamine modulating compound, alone or combination with another therapy, continuously for a prolonged period of time, such that on time is increased in the subject. As used herein, in a subject suffering from a dopamine associated state, e.g., Parkinson's Disease, the term “on time” refers to the time in which an administered dopamine modulating compound is therapeutically effective in the subject. In some embodiments, on time includes periods of time in which the subject has no or little bradykinesia or dyskinesia. In another embodiments, on time includes periods of time in which the subject has a Clinical Rating Scale of less than about 7.

In some embodiments, off time is reduced, the severity of off time symptoms are reduced and/or the frequency of off time symptoms are reduced. As used herein, in a subject suffering from a dopamine associated state, e.g., Parkinson's Disease, the term “off time” refers to the time in which an administered dopamine modulating compound is not therapeutically effective in the subject. In some embodiments, off time includes periods of time in which the subject has noticeable bradykinesia or dyskinesia. In other embodiments, off time includes periods of time in which the subject has a Clinical Rating Scale of greater than about 7.

In some embodiments, the incidence of motor response complications are reduced. In yet other embodiments, there are no periods of hyperkinetic activity or there are no significant and/or prolonged periods of hyperkinetic activity. In some embodiments, the subject does not experience any hyperkinetic symptoms or disorders. In some embodiments, the subject does not experience any paralysis.

In some embodiments, the invention includes methods of treating Parkinson's Disease or related disorders without the side effects associated with administration of a dopamine modulating compound by pump infusion.

In some embodiments, the dopamine modulating compound is administered such that normal patterns of activity are substantially restored in the subject for at least 12 hours per day. In some embodiments, the dopamine modulating compound is administered such that normal patterns of activity are substantially restored in the subject for at least 14 hours per day. In some embodiments, the dopamine modulating compound is administered such that normal patterns of activity are substantially restored to in the subject for at least 16 hours per day. In some embodiments, the dopamine modulating compound is administered such that normal patterns of activity are substantially restored in the subject for at least 18 hours per day. In some embodiments, the dopamine modulating compound is administered such that normal patterns of activity are substantially restored in the subject for at least 20 hours per day. In some embodiments, the dopamine modulating compound is administered such that normal patterns of activity are substantially restored in the subject for at least 22 hours per day. In some embodiments, the dopamine modulating compound is administered such that normal patterns of activity are substantially restored in the subject for 24 hours per day.

In some embodiments, the dopamine modulating compound is administered such that normal patterns of activity are substantially restored in the subject immediately after waking up from sleep. In some embodiments, the dopamine modulating compound is administered such that normal patterns of activity are substantially restored in the subject immediately after waking up from sleep, and such normal patterns of activity continue for at least about 18 hours. In one embodiment of the invention the subject is capable of normal activity during sleep. In yet another embodiment of the invention the patient is capable of normal movement or activity continuously.

In some embodiments, the subject experiences a period of on time immediately after waking up from sleep. In some embodiments, the subject experiences a period of on time immediately after waking up from sleep which lasts for at least 12 hours, at least 14 hours, at least 16 hours, at least 18 hours, at least 20 hours, at least 22 hours, or 24 hours.

In some embodiments, sustained efficacy is achieved in the subject for greater than 15 days, greater than 30 days, greater than 45 days, greater than 60 days, greater than 75 days, greater than 90 days, or more. In one embodiments, sustained efficacy is achieved in the subject for greater than about 30 days. In one embodiments, sustained efficacy is achieved in the subject for greater than about 60 days.

In general, the dosages needed in practicing the methods described herein are less than typical oral dosages. In some embodiments, the delivery dose required to achieve the same pharmacokinetic profile as an orally administered dose is 1/9^{th} or 1/18^{th} that of the approved orally administered dose.

In one embodiment of the invention the dopamine modulating compound is delivered via an implant or via a depot. In some embodiments, the implant includes a to core and a sheath as described in more detail herein.

In a further embodiment, the invention also features a method for treating a subject for a dopamine associated state. This method includes administering to a subject a biodegradable implant of the invention. The biodegradable implant is comprised of one or more of biodegradable implant sections and releases an effective amount of a dopamine modulating compound over a treatment period, such that said subject is treated for the dopamine associated state.

The term “dopamine associated state” includes states which can be treated by the administration of a dopamine modulating compound or otherwise associated with the presence or absence of dopamine. Examples of dopamine associated states include Parkinson's disease, attention deficit disorder (ADD), attention deficit hyperactivity disorder (ADHD), autism, pervasive development disorder (PDD), Asberger's syndrome, toxin-induced Parkinsonism, disease-induced Parkinsonism, erectile dysfunction, restless leg syndrome, and hyperprolactinemia. The term “Parkinsonism” includes conditions resulting from injury to the central nervous system that may cause an individual to exhibit symptoms similar to those of Parkinson's disease. Parkinsonism may result, for example, from toxin exposure, for example, carbon monoxide or manganese poisoning or 1-methyl-4-phenyl-1,2,3,6-tetrahydropyridine hydrochloride (“MPTP”) administration, or from a disease condition such as encephalitis. In some embodiments, the dopamine associated state is Parkinson's Disease. In some embodiments, the dopamine associated state is mild to moderate Parkinson's Disease.

The dopamine modulating compound concentrations may range from about 5% to about 95%, from about 10% to about 80%, from about 20% to about 60%, from about 40% to about 60%, from about 45% to about 55%, or about 50% in the implant depending upon the release period.

The term “subject” includes animals (e.g., mammals, e.g., cats, dogs, horses, pigs, cows, sheep, rodents, rabbits, squirrels, bears, primates (e.g., chimpanzees, gorillas, and humans)) which are capable of (or currently) suffering from dopamine associated states. It also includes transgenic animal models. In a further embodiment, the subject is a human suffering from Parkinson's disease or disease or toxin induced Parkinsonisms.

The term “treated,” “treating” or “treatment” includes therapeutic and/or to prophylactic treatment of a dopamine associated state. The treatment includes the diminishment or alleviation of at least one symptom associated or caused by the dopamine associated state. For example, treatment can be diminishment of one or several symptoms of the dopamine associated state or complete eradication.

The language “effective amount” of the dopamine modulating compound is that amount necessary or sufficient to treat or prevent a dopamine associated state in a subject, e.g. prevent the various morphological and somatic symptoms of a dopamine associated state in a subject. The effective amount can vary depending on such factors as the size and weight of the subject, the type of illness, or the particular dopamine modulating compound. For example, the choice of the dopamine modulating compound can affect what constitutes an “effective amount.”

The term “effective amount” also includes the amount of the dopamine modulating compound that will render a desired therapeutic outcome, e.g., a level or amount effective to reduce symptoms of a dopamine associated state such as Parkinson's disease and/or increase periods of therapeutic effectiveness (“on” periods) for a patient undergoing chronic dopaminergic therapy for idiopathic Parkinson's disease or toxin- or disease-induced Parkinsonism, or beneficial treatment, i.e., reduction or alleviation of adverse or undesirable symptoms of a condition treatable with a dopamine agonist, such as erectile dysfunction, restless leg syndrome, or hyperprolactinemia. For treatment of Parkinson's disease or Parkinsonism, effectiveness is often associated with reduction in “on”/“off” fluctuations associated with a particular Parkinson's disease treatment regime, such as for example, chronic levodopa administration. An amount that is “therapeutically effective” for a particular subject may depend upon such factors as a subject's age, weight, physiology, and/or the particular symptoms or condition to be treated, and will be ascertainable by a medical professional.

In a further embodiment, the effective amount of the dopamine modulating compound is the amount necessary to achieve a plasma concentration of the dopamine modulating compound of about 0.5 ng/mL to about 100 ng/mL, of about 0.5 ng/mL to about 90 ng/mL, of about 0.5 ng/mL to about 80 ng/mL, of about 0.5 ng/mL to about 70 ng/mL, of about 0.5 ng/mL to about 60 ng/mL, of about 0.5 ng/mL to about 50 ng/mL, about 1 ng/mL to about 40 ng/mL, about 1 ng/mL to about—30 ng/mL, about 1 ng/mL to about 20 ng/mL, about 1 ng/mL to about 15 ng/mL, or about 2.5 ng/mL to about 10 to ng/mL. In a further embodiment, the effective amount is effective to maintain the aforementioned plasma concentration for at least one day or longer, one week or longer, two weeks or longer, three weeks or longer, four weeks or longer, six weeks or longer, two months or longer, three months or longer, four months or longer, five months or longer, six months or longer, seven months or longer, eight months or longer, nine months or longer, ten months or longer, eleven months or longer, twelve months or longer, or over a year or longer. In some embodiments, the release period is about 40 to about 80 days, from about 50 to about 70 days or about 60 days.

Accordingly, in some embodiments, the implant sections of the present invention are able to maintain a plasma concentration of at least about 5 ng/mL for at least about 30 days. In some embodiments, the implant sections of the present invention are able to maintain a plasma concentration of at least about 5 ng/mL for at least about 35 days, e.g., at least about 35 days, at least about 40 days, at least about 45 days, at least about 50 days, at least about 55 days, at least about 60 days, at least about 65 days, at least about 70 days, at least about 75 days or at least about 80 days. In some embodiments, the implant sections of the present invention are able to maintain a plasma concentration of at least about 10 ng/mL for at least about 25 days, e.g., at least about 30 days, at least about 35 days, at least about 40 days, at least about 45 days.

The term “administering” includes surgically administering, implanting, inserting, or injecting the implant (or section(s) thereof) into a subject. The implant (or section) can be located subcutaneously intramuscularly, or located at another body location which allow the implant to perform its intended function. Generally, implants (or sections) are administered by subcutaneous implantation at sites including, but not limited to, the upper arm, back, or abdomen of a subject. Other suitable sites for administration may be readily determined by a medical professional. Multiple implants or sections may be administered to achieve a desired dosage for treatment.

The invention also pertains to methods comprising administering second agents in combination with the biodegradable implants of the invention. The second agents may be, for example, any agent which enhances or increases the effectiveness of the treatment of the dopamine associated state and/or reduce inflammation at the site of administration of the biodegradable implant, or which prevents or retards oxidation of the dopamine modulating compounds. For example, an anti-inflammatory agent, such as for example, a steroid (e.g., dexamethasone, triamcinolone, betamethasone, to clobetasol, cortisone, hydrocortisone, or a pharmaceutically acceptable salt thereof), or a nonsteroidal anti-inflammatory agent (“NSAID;” e.g., diclofenac potassium diclofenac sodium, diclofenac sodium with misoprostol, diflunisal, etodolac, fenoprofen calcium, flurbiprofen, ibuprofen, indomethacin, ketoprofen, meclofenamate sodium, mefenamic acid, meloxicam, nabumetone, naproxen, naproxen sodium, oxaprozin, piroxicam, sulindac, tolmetin, COX-2 inhibitors (e.g., celecoxib, rofecoxib, valdecoxib), acetylated salicylates (e.g., aspirin), nonacetylated salicylates (e.g., choline, magnesium, and sodium salicylates, salicylate)), and/or an antihistamine (e.g., loratadine (“LT”), astemizole, cetrizine dihydrochloride, chlorpheniramine, dexochlorpheniramine, diphenhydramine, mebhydrolin napadisylate, pheniramine maleate, promethazine, or terfenadine). The second agents may be encapsulated within the biodegradable implant to prevent or reduce local inflammation at the site of administration. The second agents may also be administered separately to the subject by any route that allows the second agents to perform their intended functions. The second agents may be administered orally, parentally, topically, subcutaneously, sublingually, etc. Any of the second agents, or a combinations thereof, may also be included in the same implant(s) as dopamine modulating compounds (e.g., in the core and/or in one or more sheath layers) or alternatively, may be incorporated into one or more separate implants or sections thereof that do not include the dopamine modulating compound. An antioxidant, e.g., ascorbic acid, sodium metabisulfite, glutathione, may be included in the same implant or section thereof as dopamine modulating compound to prevent or reduce oxidation of dopamine modulating compound during preparation, storage, and/or administration of the implant or section thereof.

In some embodiments, the dopamine modulating compound is co-administered with another therapy selected from dopamine metabolic inhibitors, monoamine oxidase inhibitors, dopaminergetics, dopamine agonists or adenosine receptor antagonists. In some embodiment, the co-administered therapy is a dopaminergic, i.e. L-Dopa.

In yet another embodiment of the invention the amount co-administered therapy needed for efficacy is significantly less and/or the side effects corresponding to the co-administered therapy are significantly reduced.

In still other embodiments, the present invention provides methods for the treatment of Parkinson's Disease in a patient in need thereof. Such methods include to administering a continuous and prolonged delivery of Ropinirole via an implant, in combination with L-Dopa, wherein on time is increased and off time is decreased.

The term “implant” includes surgically implantable devices comprised of one or more sections. The sections may be of any size which allows the implant to perform its intended function. In one embodiment, the sections and/or implant are removable from the subject. In another embodiment, the implant is comprised of 1, 2, 3, 4, 5, 6, 7, 8, 9, 10, or more discrete sections.

In another embodiment, the section may be rod shaped or any other shape which allows for the implant to perform its intended function. The term “rod shaped” includes shapes which are about cylindrical. The sections of the present may also be formed with any cross-sectional geometry, e.g., a circle, an ellipsoid, a lobe, a square, or a triangle.

In one embodiment, the sections are macroscopic (e.g., at least 1 mm in diameter). In a further embodiment, the sections are rod shaped.

In one embodiment, the invention pertains to a cylindrical rod shaped biodegradable implant section. The implant section comprises a core and at least one sheath. In some embodiments, the core includes a dopamine modulating compound and a biodegradable polymer. In some embodiments, the sheath includes a biodegradable polymer. In some embodiments, the sheath consists essentially of a biodegradable polymer. Optionally, each end of the rod shaped implant section is also coated with a biodegradable polymer.

Accordingly, in some embodiments, the invention pertains to a rod shaped biodegradable implant section, which includes a core which comprises a dopamine modulating compound and a first biodegradable polymer, and a sheath which comprises a second biodegradable polymer. In some embodiments, the sections of the present invention further include a third biodegradable polymer on one or both ends of the section. Such third biodegradable polymer can be coated onto one or both ends of the section using, for example, dip coating.

As used herein, the term “core” refers to the central portion of the implant as examined at a cross section. As used herein, the term “sheath” refers to an outer coating material situated around the core material. The sheath extends inwardly from the outside perimeter of the implant into a portion of the overall cross-sectional area of the implant section. In some embodiments, the sheath is a continuous coating, e.g., a layer or layers. In some embodiments, the sheath provides a uniform, continuous coating to around the entire perimeter of the core.

In some embodiments, the implant sections of the present invention also exhibit superior in vivo and in vitro release characteristics. For example, in some embodiments, the sections of the present invention exhibit little or no initial burst upon contact with a biological or aqueous medium. As used herein, the phrase “little or no initial burst” refers to an amount of compound released 24 hours subsequent to the initial quantifiable release which is no greater than the steady state amount of compound release over the effective lifetime of the implant. In other embodiments, the sections of the present invention exhibit little or no lag upon contact with a biological or aqueous medium.

In some embodiments, the implant sections of the present invention release substantially all of the dopamine modulating compound during the effective lifetime of the implant. In some embodiments, the sections release at least about 60% of the dopamine modulating compound upon contact with a biological or aqueous medium. In some embodiments, the sections release at least about 70%, e.g., at least about 80%, at least about 85%, at least about 90% or at least about 95% of the dopamine modulating compound upon contact with a biological or aqueous medium. In some embodiments, the sections release about 100% of the dopamine modulating compound upon contact with a biological or aqueous medium. In still further embodiments, the implant sections of the present invention exhibit substantially linear release of the dopamine modulating compound upon contact with a biological or aqueous medium.

It was found, using the formulations of the invention, that the initial amounts of dopamine modulating compound released into the subject are low (e.g., less than the effective amount) and less than the amount targeted and achieved during steady state. The implant sections described in this application reliably release the dopamine modulating compound to the subject. The amount of dose variation is very low (e.g., less than about ±20%, less than about ±10%, or less than about ±5%). The low amount of variation (and the substantial lack of an initial burst) allows the implants of the invention to administer sufficient amounts of the dopamine modulating compounds to achieve therapeutic effects (e.g., reduction of bradykinesia or treatment of the dopamine associated state) without significant undesirable side effects (e.g., psychomotor agitation or psychosis).

The sections of the present invention, e.g., those prepared by continuous extrusion process, may be cut or otherwise made any desired length, e.g., for dosing to and/or ease of handling. Moreover, a long piece of extruded material may be maintained, e.g., rolled onto a spool or coil or maintained in longer pre-determined lengths, prior to cutting the material into a size suitable for implantation. The sections may also be prepared in a variety of diameters depending, e.g., on the total dose of drug. In another embodiment, the sections are about 0.5 mm to about 5 mm in diameter and about 0.5 cm to about 10 cm in length. In a further embodiment, the sections are about 0.5 mm to about 5 mm in diameter and about 0.5 cm to about 5 cm in length. In another further embodiment, the sections are about 1 mm to about 3 mm in diameter and about 1 cm to about 3 cm in length.

In a further embodiment, the implant sections comprised of a biocompatible and/or biodegradable polymer. Preferably, the implant sections are removable throughout the time period when the dopamine modulating compound is being released to the subject at therapeutic levels.

The term “biodegradable” includes polymers which degrade (e.g., chemically, physically, enzymatically, etc.) by bodily processes to products readily excreted by the body and, advantageously, do not accumulate in the body. The products of the biodegradation should also be biocompatible with the body in the same sense that the polymeric matrix is biocompatible with the body. Suitable examples of biodegradable polymers include poly(glycolic acid), poly-D,L-lactic acid, poly-L-lactic acid (PLA), copolymers of the foregoing (e.g., poly(lactide-co-glycolide) (PLGA), e.g., 85:15 PLGA, 75:25 PLGA, 50:50 PLGA, etc.), poly(aliphatic carboxylic acids), copolyoxalates, polycaprolactone (PCL), polydioxonone, poly(ortho carbonates), poly(acetals), poly(lactic acid-caprolactone), polyorthoesters, poly(glycolic acid-caprolactone), polyanhydrides, polyhydroxy acids, polyetheresters, polyethylene glycol, polyesteramides, polyphosphazines, polycarbonates, polyamides and copolymers and blends thereof as well as natural polymers including polysaccharides, proteins, albumin, casein, and waxes, such as, glycerol mono- and distearate, and the like. Furthermore, some polymers may also be modified with end cap modifications such as alkyl caps. Such end caps are described in Journal of Controlled Release 52 (1998) 53-62 and Journal of Controlled Release 67 (2000)281-292, the contents of each of which are incorporated herein by reference. In some embodiments, the biodegradable polymer is a biodegradable aliphatic polyester. In some embodiments, the biodegradable polymer is a non-saccharide polymer.

In one embodiment, the implant is comprised of a polymer that is biocompatible. The term “biocompatible” includes polymers which are not toxic to the human body, are not carcinogenic, and do not significantly induce inflammation in body tissues.

In one embodiment, the polymer comprises polylactide or a copolymer comprising polylactide such as dl(polylactide-co-glycolide). Examples of such biodegradable polymers include those which comprise about 30 mole % to about 100 mole % polylactide and about 0 mole % to about 70 mole % polyglycolide. Any value or range intermediate to the recited range is meant to be encompassed by the present invention. For example, in some embodiments, the biodegradable polymers include about 30% polylactide and about 70% polyglycolide. In further embodiments, the biodegradable polymers include about 40%, e.g., about 50%, about 60%, about 70%, about 80%, about 90% or about 95% polylactide. In still further embodiments, the biodegradable polymers include about 60%, e.g., about 50%, about 40%, about 30%, about 20%, about 10% or about 5% polyglycolide. In a further embodiment, the biodegradable polymer is 100% PLA.

The implant sections of the invention may comprise a biodegradable coating (optionally hydrophobic) on each end of the rod shaped implant section. The biodegradable end coating includes a third biodegradable polymer, which may be any biodegradable polymer described herein. Non-limiting examples of such biodegradable polymers include poly(lactide-co-glycolide) (PLGA) (including but not limited to 85:15 PLGA, 75:25 PLGA, 50:50 PLGA, etc.), polycapralactone (PCL), PLA, and combinations and co-polymers thereof (including, but not limited to, PLGA-co-PCL and PLA-co-PCL). The biodegradable coating may be applied to each end of the implant section by dip coating the each end of the implant section in a solution of the polymer (e.g., a 10% PLA solution). The biodegradable coating may optionally be formed on one or both ends by any method known in the art, including those described in more detail infra. In a further embodiment, the biodegradable coating is PLA.

Accordingly, in some embodiments, the first biodegradable polymer (e.g., the “core” polymer) includes PLA, e.g., 100 mole % poly-DL-lactide having a target inherent viscosity (IV) range of 0.55-0.85 dL/g. In some embodiments, the second biodegradable polymer (e.g., the “sheath” polymer) includes PLA, e.g., 100 mole % poly-DL-lactide having a target IV range of 0.35-0.65 dL/g. In some embodiments, the third biodegradable polymer (e.g., the “end” polymer) comprises PLA and optionally PLGA, e.g., about 50-100% by weight of 100 mole % poly-DL-lactide having a target IV range of 0.35-0.65 dL/g and about 0-50% by weight of poly(DL-lactide-co-glycolide) having a target IV range of between about 0.50 and about 0.76 dL/g. In some embodiments, the target IV is about 0.50 dL/g. In some other embodiments, the target IV is about 0.76 dL/g.

One of skill in the art would be able with no more than routine experimentation, to determine the target inherent viscosity of the biodegradable polymer, for example, by a glass capillary viscometer. In some embodiments, the measurement of the target inherent viscosity is performed in chloroform at 30° C. with a concentration of 500 mg polymer dissolved in 100 mL of solvent.

In another embodiment, the invention pertains, at least in part, to a biodegradable implant section which includes a core and two or more coating layers. In some embodiments, the core comprises a dopamine modulating compound and a biodegradable polymer, and the sheaths comprise independently selected amounts of a dopamine modulating compound and a biodegradable polymer.

The polymers used in each of these sheaths may be different, along with different amounts of dopamine modulating compound in each layer. The dopamine modulating compound loading in the implant section may be between about 0.1 wt % and about 80 wt %, e.g., between about 1 wt % and about 70 wt %, e.g., between about 10 wt % and about 60 wt %, e.g., between about 20 wt % and about 50 wt %. In some embodiments, the amount of dopamine modulating compound in each individual sheath may vary from about 0% to about 50% by weight. In some embodiments, one or more sheaths include no dopamine modulating compound. The identity of the polymer and the amount of drug in each layer may be independently selected such that a particular delivery profile is achieved. Preferably, the implant sections of the invention are formulated such that there is no significant “initial burst” of dopamine modulating compound when administered to the subject.

The term “dopamine modulating compound” includes both dopamine agonists and antagonists. In a further embodiment, the dopamine modulating compound is a dopamine agonist. Examples of dopamine agonists include compounds which are capable of binding to one or more dopamine receptor subgroups, resulting in beneficial therapeutic effect in an individual treated with the agonist. The dopamine agonists may be agonists for at least the D2 subgroup of dopamine receptors, and also may be agonists to for D1 and/or D3 receptors. Examples of dopamine modulating compounds of the invention include apomorphine, lisuride, pergolide, bromocriptine, pramipexole, 4-alkylamino-2(3H)-indolone compounds (e.g., ropinirole), rotigotine, docarpamine, terguride, cabergoline, levodopa, spheramine, romergoline, carmoxirole, zelandopam, sumanirole, sibenadet, and combinations of two or more of these dopamine agonists. Pharmaceutically acceptable salts, esters, prodrugs, and metabolites of these compounds are also included. In one further embodiment, the dopamine agonist is ropinirole. In some embodiments, the dopamine agonist is not apomorphine.

The term “4-alkylamino-2(3H)-indolone compound” includes compounds of the formula (I):

wherein:

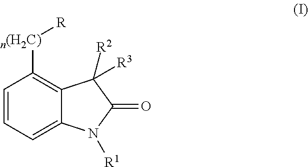

R is amino, alkylamino, di-alkylamino, alkenylamino, dialkenylamino, N-alkyl-N-alkenylamino, benzylamino, dibenzylamino, arylalkylamino, or diarylalkylamino;

R¹, R² and R³ are each independently hydrogen or alkyl; and

n is 1, 2, or 3, and pharmaceutically acceptable salts thereof.

In a further embodiment, R is 4-hydroxyphenethylamino or di-(4-hydroxyphenethylamino). In another further embodiment, R is amino, di-n-propylamino, n-propyl-n-butylamino or 4-hydroxyphenethylamino. In an embodiment, R¹, R², and R³ are each lower alkyl (e.g., 1-6 carbons). In another further embodiment, R¹, R², and R³ are each hydrogen. In yet another further embodiment, n is 2. In one embodiment, the compound of formula (I) is 4-(2-di-n-propylaminoethyl)-2(3H)-indolone (“ropinirole”) or a pharmaceutically acceptable salt thereof.

The term “lower alkyl” includes branched and straight chain groups of from 1-6 carbons, preferably methyl, ethyl, propyl, or butyl for each alkyl in R and from 1-4 carbons, preferably methyl, for each of R¹, R² and R³.

Pharmaceutically acceptable acid addition salts of the dopamine modulating compounds are also part of this invention. The salts are prepared by methods well known to the art and are formed with both inorganic or organic acids, for example: to maleic, fumaric, benzoic, ascorbic, pamoic, succinic, bismethylenesalicylic, methane sulfonic, ethane disulfonic, acetic, oxalic, propionic, tartaric, salicylic, citric, gluconic, aspartic, stearic, palmitic, itaconic, glycolic, p-aminobenzoic, glutamic, benzenesulfonic, hydrochloric, hydrobromic, sulfuric, cyclohexylsulfamic, phosphoric and nitric acids. The hydrohalic salts also may be used.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxophenyl, quinoline, isoquinoline, naphthridine, indole, benzofuran, purine, benzofuran, deazapurine, or indolizine. Those aryl groups having heteroatoms in the ring structure may also be referred to as “aryl heterocycles,” “heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or to phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls,” the latter of which refers to alkynyl moieties having substituents to replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” to includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively.

The term “amide” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The term “heteroatom” includes atoms of any element other than carbon or hydrogen. Preferred heteroatoms are nitrogen, oxygen, sulfur and phosphorus.

The term “cyclic” includes saturated or unsaturated, aromatic or non-aromatic ring moieties. Examples of saturated cyclic moieties include piperidine, piperazine, morpholine, cyclohexyl, cyclobutyl, cyclopentyl, etc.

The alkylated products may be prepared by alkylation of the parent amino compounds of formula I in which R is amino or a secondary amino. For example, the N-alkylated products, formula I when R is a secondary or tertiary amino, are conveniently prepared by reductive alkylation using, for example, the aldehyde in one or two molar equivalent quantities under reduction conditions, such as under catalytic hydrogenation conditions over a palladium or platinum catalyst or such as using formaldehyde-formic acid when R is dimethylamino.

N-Alkylation, such as using an allyl or benzyl halide in the presence of an acid binding agent, can be used under standard mild conditions. Protecting the amido hydrogen in the ring is also used during alkylation if necessary as known to the art. Alkyl substituents at the 1 or 3-positions of the indolone ring are introduced by forming the lithio derivatives at the ring position, such as using butyl lithium, followed by reaction with a lower alkyl halide, especially an alkyl iodide.

In another embodiment, the invention also features a rod shaped biodegradable implant section which includes about 45%-55% by weight ropinirole and about 45%-55% by weight PLA. In some embodiments, each end of said rod shaped biodegradable implant section is coated with PLA.

In yet another embodiment, the invention also includes a rod shaped biodegradable implant section which consists essentially of about 45%-55% by weight ropinirole and about 45%-55% by weight PLA, wherein each end of said rod shaped biodegradable implant section is coated with PLA.

The implants (and sections thereof) can be manufactured using methods known in the art. See, for example, US Patent Application No. 20030007992; US Patent Application No. 20060159721; Cowsar and Dunn, Chapter 12 “Biodegradable and Nonbiodegradable Delivery Systems” pp. 145-162; Gibson, et al., Chapter 31 “Development of a Fibrous IUD Delivery System for Estradiol/Progesterone” pp. 215-226; Dunn, et al., “Fibrous Polymers for the Delivery of Contraceptive Steroids to the Female Reproductive Tract” pp. 125-146; and Dunn, et al., “Fibrous Delivery Systems for Antimicrobial Agents” from Polymeric Materials in Medication ed. C. G. Gebelein and Carraher (Plenum Publishing Corporation, 1985) pp 47-59.

For example, in some embodiments, an implant of the present invention is manufactured by extrusion molding. In one embodiment, the extrusion molding is high-pressure extrusion molding. Each method of manufacture may provide one or more beneficial properties, e.g., increased density, uniformity, variety of shapes, low material loss, etc.

In some embodiments, the implants and/or sections of the present invention are formed via coaxial extrusion. With coaxial extrusion, a first polymeric matrix (e.g., including one or more dopamine modulating compounds) is extruded as the core at substantially the same time as the second polymeric matrix is extruded as the to membrane/sheath. A typical coaxial apparatus consists of two or more concentric rings. The first polymeric matrix is pumped through the inner ring, where it forms the core. The second polymeric matrix (and other additional polymeric matrices) is pumped through the outer ring(s) to form the sheath(s). The relative diameters of the core and sheath may be controlled, e.g., by the dimensions of the die, the extrusion conditions, the relative extrusion rates of the two extruders, and the relative take-off speed. Accordingly, in some embodiments, the core diameter and membrane thickness are independently controlled. Additional methods for preparing coaxial implants are known in the art.

In some embodiments, the section of the present invention is a coaxial, rod shaped biodegradable implant section. That is, in some embodiments, the sections of the present invention are manufactured using co-axial extrusion techniques. Without wishing to be bound by any particular theory, it is believed that coaxial implant sections exhibit certain surface properties, such as those described herein. It is believed that such properties, in turn, lead to desirable release characteristics.

In some embodiments, the implants of the present invention exhibit enhanced surface roughness characteristics, e.g., versus uncoated implants or dip-coated implants. As used herein, the phrase “surface roughness” refers to the measure of the fine irregularities on the surface of the implants of the present invention. Surface roughness may be calculated, for example, as the mean of the absolute values of the surface departures from the mean plane. In some embodiments, the implant sections of the present invention exhibit a surface roughness which is greater than about 1.5 μm. For example, in some embodiments, the surface roughness of the section is greater than about 2.0 μm, greater than about 2.5 μm, greater than about 3.0 μm, greater than about 3.5 μm, greater than about 4.0 μm or greater than about 4.5 μm. In some embodiments, the surface roughness of the section is between about 1.5 μm and about 4.5 μm, e.g., between about 2.0 μm and about 3.5 μm.

In some embodiments, the implants of the present invention exhibit enhanced percent porosity, e.g., versus uncoated implants or dip-coated implants. As used herein, the phrase “percent porosity” refers to the average percent of the interior space of the implant of the present invention which is occupied by void spaces or pores. Accordingly, in some embodiments, the implant sections of the present invention exhibit a percent porosity of about 1.5% to about 3.5%. In some embodiments, the implant to sections of the present invention have a percent porosity of about 1.75% to about 3.25%, e.g., about 2.0% to about 3.0%.

In some embodiments, the implants of the present invention exhibit enhanced surface pore depth, e.g., versus uncoated implants or dip-coated implants. As used herein, the phrase “surface pore depth” refers to an average peak to valley distance on the surface of an implant section. “Surface pore” as used herein, refers to open pores on the surface of the implant section. Accordingly, in some embodiments, the implant sections of the present invention exhibit an average surface pore depth of at least about 60 μm, e.g., at least about 65 μm. In some embodiments, the sections of the present invention have an average surface pore depth of between about 60 μm and about 100 μm.

In some embodiments, the sections of the present invention have an average surface pore depth of between about 60 μm and about 90 μm, e.g., between about 65 μm and about 90 μm, e.g., between about 65 μm and about 80 μm.

In some embodiments, exposed ends of the core are sealed, e.g., with a third polymeric matrix (which may be the same or different than the second polymeric matrix used in the sheath). The polymer utilized in the third polymeric matrix may be, e.g., any of the biodegradable polymers described herein. In some embodiments, however, only one or neither of the exposed ends are sealed, e.g., so that an initial loading dose may be released from the core. Several methods can be used to seal the ends of the implants, including, but not limited to coating with a solution of the sheath polymer, applying molten sheath polymer, cutting the implant with a hot knife or wire such that it is heat sealed as the cut is made, and/or placing a polymer plug into the end of the implant.

In some embodiments, the thickness of the sheath will be between about 2% and about 40% of the overall implant diameter, e.g., between about 5% and about 30% of the total diameter. The sheath polymer may be dense and have little or no porosity or it may be highly porous having pores of about 1 to about 30 microns and pore volumes of between about 5% and about 70%. The sheath polymer may also contain the dopamine modulating compound at a lower loading than is contained in the core, or it may contain a different active ingredient than is contained in the core. In some embodiments, however, little or no dopamine modulating compound is contained within the sheath.

The dopamine modulating compound can be added to the formulation, e.g., by mixing to form a slurry, by solvent-blending, dry blending, and/or melt blending with the polymeric matrix. Uniform mixing may be obtained by extruding the drug-matrix twice. In some embodiments, the core is formulated by dry blending the dopamine modulating compound and polymer, melt extruding the blend, and grinding the extrudate to be used for a second extrusion.

For implants comprised of polymers that are viscose liquids at processing temperatures of 60-80° C. (e.g., polycaprolactone and the like), the polymer is melted in an oven, oil bath or by another method known in the art, and the dopamine modulating compound is mixed into the molten polymer with an electric mixer. The homogenous mixture of the dopamine modulating compound and the polymer is then formed into implants by extrusion.

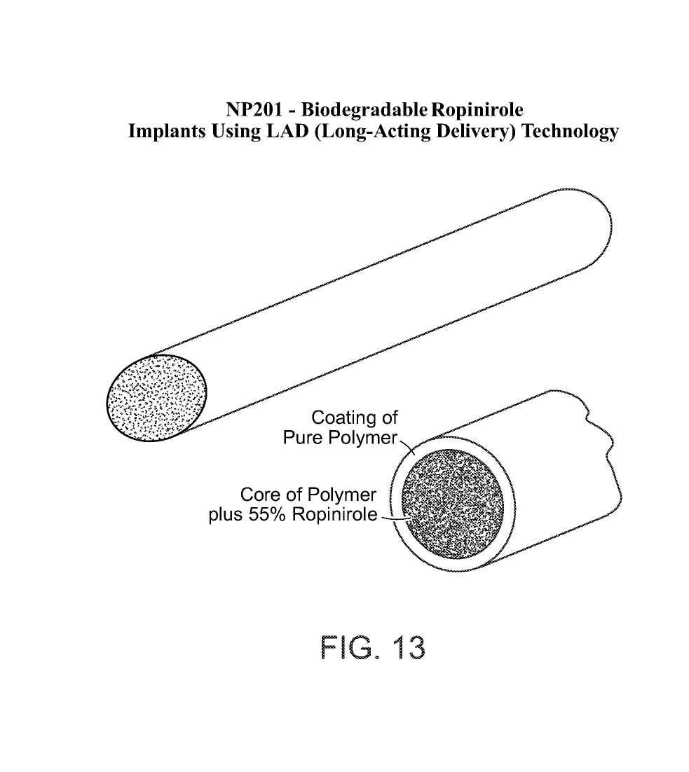

For implants (or sections thereof) comprised of polymers that require pressure to flow at processing temperature, the dopamine modulating compound and the polymer are melt mixed in a single or twin screw mixer/extruder that heats and kneads the drug and polymer prior to extrusion. The implants (or sections thereof) are then formed by extrusion alone or in combination with compression molding. The implants may further be dip coated with a polymer solution (e.g., 100% PLA). The implants may be entirely dip coated or only dip coated on each end of the rod shape, as shown in FIG. 13.

For implants (or sections thereof) comprised of polymers and/or dopamine modulating compounds which are solvent blended, e.g., prior to extrusion, the selection of the solvent used in the process generally depends on the polymer and active agent chosen, as well as the particular means of solvent removal to be employed. Such solvents are known to the skilled artisan, however, non-limiting examples include organic solvents, such as acetone, methyl ethyl ketone, tetrahydrofuran, ethyl lactate, ethyl acetate, dichloromethane, and ethyl acetate/alcohol blends.

### Exemplification of the Invention

Ropinirole, a D2 dopamine agonist that acts on D2 postsynaptic receptors, has been shown to be effective in treating Parkinson's symptoms in randomized, placebo controlled studies. This study compared two different ways to administer ropinirole in 1-methyl-4-phenyl-1,2,3,6-tetrahydropyridine hydrochloride (“MPTP”) treated monkeys. The hypothesis was that monkeys receiving ropinirole orally will return to Parkinsonian state (e.g., bradykinesia, freezing, stooped posture and tremor) sooner than monkeys receiving an exemplary ropinirole subcutaneous implant of the present invention (“NP201”). Following induction of Parkinsonian symptoms with MPTP, to monkeys were treated with either oral or implanted ropinirole to compare pharmacokinetic parameters and symptomatic control. Objectives included comparison of the efficacy of NP201 with oral ropinirole in treating Parkinsonian rhesus monkeys, a primate model of Parkinson's Disease, assessment of plasma levels of thrice daily (“TID”) oral ropinirole and NP201, and measurement of the degree of reduction in bradykinesia and dyskinesia in oral administration and NP201 administration.

The study was performed in four phases: Quarantine Phase, Training Phase, MPTP Induction Phase, and Dose Finding Phase/Experimentation Phase. In the Quarantine Phase, monkeys were admitted and quarantined for a period of 30 days. While in quarantine, monkeys were monitored daily for food intake, general behavior and appearance. Monkeys were acclimatized for a period of at least 48 hours after their arrival. In the Training Phase, monkeys were trained to perform the Fine Motor Pick-up Test. The Clinical Rating Scale was completed in order to obtain baseline data.

In the MPTP Induction Phase, the monkeys were made bilaterally Parkinsonian with the use of MPTP. Monkeys received an initial dose of 0.3 mg/kg MPTP through a unilateral intracarotid injection and twice weekly injections of 0.3 mg/kg intravenous (i.v.) MPTP until they achieve a score of between 7.0 and 20.5 (e.g., 12 or higher) on the clinical rating scale with moderate bilateral Parkinsonism. Intravenous MPTP is administered at 3-5 day intervals. The actual cumulative number of i.v. MPTP injections is determined by the onset of stable bilateral symptoms. This protracted time course of i.v. MPTP administration allowed for development and evaluation of new Parkinsonian symptoms following each MPTP administration. Ninety-six hours after MPTP, before release from quarantine, Parkinsonian disability ratings were done on each monkey. Endpoint criteria for terminating administration was a bilateral Parkinsonian syndrome characterized by a Clinical Rating Scale (“CRS”) score of 12 or more. Monkeys that become appropriately bilaterally Parkinsonized, as evidenced by a Clinical Rating Score between 12 to 20 after MPTP i.v. administration, were included in the study. Monkeys who score ≦11 or ≧21 on the Clinical Rating Score were excluded. Monkeys who lose 20% of their body weight over the course of the study were discontinued from the study. Body weight was checked weekly.

In the Dose Finding Phase, all monkeys were dosed to determine the lowest effective dose of ropinirole per monkey. The lowest effective dose was defined as the dose that causes a 50% reduction in the Clinical Rating Scale score. All monkeys to received escalating doses of oral ropinirole as follows to determine each animal's lowest effective oral dose: 1.0 mg/kg in the morning followed by PK blood samples (day 1); 0.25 mg/kg TID for three days (days 2-4); 0.50 mg/kg TID for three days (days 5-7); 1.00 mg/kg TID for three days (days 8-10); 2.00 mg/kg TID for three days (days 11-13); and 1.0 mg/kg in the morning followed by PK blood samples (day 14)

If no dose achieved 50% reduction of Clinical Rating score, titration was continued the following week. The mean dose was used to determine the study comparison dose for implants. The lowest effective dose was administered TID throughout the study in Group 1 of the Experimentation Phase.

In the Experimentation Phase, the monkeys were assigned to one of three treatment groups: Group 1—oral ropinirole TID using the mean lowest effective dose determined in the Dose Finding Phase. Group 2—NP201 releasing ropinirole at a dose of either approximately 1/9^{th} or 1/18^{th} of the total daily oral TID dose), e.g., for a single 1.00 mg/kg oral dose, the daily dose would be 3.00 mg/kg/day and the implant doses would then be 0.33 mg/kg/day or 0.17 mg/kg/day, respectively. Group 3—Control group will receive both a subcutaneous placebo implant and an oral placebo TID.

All subjects received daily oral treatments and implants in a double dummy design to control for the effects of implantation, the presence of an implant under the skin, oral administration in fruit, and to maintain the blind for investigators.

Treatment groups were matched for the Clinical Rating Scale and Fine Motor Pick-up Test severity such that there was no statistically significant difference between groups for either measure. Monkeys were assigned to one of four severity-matched groups (n=4 monkeys in each group). The resulting 3 groups were randomly assigned to a treatment condition. Oral dosing of ropinirole and placebo was done BID one day per week when monkeys receive anesthesia for blood draws, site inspection and retrieval of activity monitor data.

While in the Experimentation Phase, monkeys were tested on the Fine Motor Pick-up Test three times per week. The Clinical Rating Scale and Global Primate Dyskinesia Scale were completed one day per week, prior to and following oral dosing. All monkeys continuously wore activity monitoring jackets throughout the study.

In order to collect Pharmacokinetic (“PK”) samples, monkeys were tranquilized with ketamine (7-10 mg/kg intramuscularly) in order to collect 1 cc of blood from the saphenous vein. Plasma samples were collected into appropriately labeled collection to tubes. One cc blood samples were collected by catheter or venipuncture into EDTA collection tubes for ropinirole plasma concentrations by a validated HPLC with MS/MS detection. The plasma samples were frozen within two hours after collection and remained frozen until analyzed. PK Samples for the Dose Finding Phase (Oral dose) were drawn on days 1 and 14 at 60, 120, 180, 240 and 360 minutes after one oral dose of 1.0 mg/kg. PK Samples for the Experimentation Phase (NP201 implants) were drawn in all four test groups in order to test the blood levels of ropinirole. Plasma samples for serial PK are drawn at the following intervals in all monkeys (ropinirole and placebo implants): 30-60 minutes and 6 hours±30 min post implantation (record time), and 8, 15, 22, 29, 36, 43, 50, 57 and 64 days after implantation of NP201.

A Clinical Rating Scale for neurobehavioral examination was used to assess the clinical status of the monkeys under; normal, MPTP, and MPTP+treatment conditions, once per week, according to previously published protocols. A trained observer blind to the treatment conditions, performed the ratings over the entire duration of the study (study days 4-82). Subjects were assessed once per week. All groups were assessed one hour after dosing. The scale consisted of the following ratings: tremor (0-3 for each arm); posture (0-3); gait (0-5); bradykinesia (0-5); balance (0-3); gross motor skills (0-4 for each arm); defense reaction (0-2); and freezing (0-2). The score was obtained as the sum of the features. Out of a total of 34 points, 0 corresponds to normal scoring and 34 to extreme severe disability. Occurrence of dyskinesia, psychological disturbances and vomiting were also recorded.

The activity monitoring system (“AMS”) records a 12-hour light/dark cycle. Each monkey was fitted with a monkey jacket that contained an activity monitor (Actitrac 1M Systems, Baltimore, Md.) in the inside back pocket. This activity monitor measured motion along its vertical and horizontal axes digitizing acceleration signals at a 40 Hertz sampling rate and stored the average acceleration value calculated during each consecutive time interval. The number of pulses was expressed for a pre-selected time period (1 minute). Data was collected continuously in 1-minute bins for a period of 14 days. At the end of the period, the monkeys were again tranquilized with ketamine (15 mg/kg, intramuscularly), the activity monitor was removed and interfaced with a computer, and the data was downloaded. The data was expressed as the mean activity of each 12-hour light/dark cycle.

In the Fine Motor Pick-up Test each monkey was tested for fine motor to performance in both upper limbs by using a modification of a food pick-up task. Testing took place in a modified home cage. The monkeys were presented with a 3×3 matrix of recessed food wells embedded in a Plexiglas board. Cubic pieces of apple (0.5 cm) were placed within each food well. During each trial, six pieces were placed within the same six food wells, and the time it takes for the monkey to retrieve all six pieces was recorded. The test board was configured in such a manner that the monkey could only retrieve the food reward by using the arm being evaluated. Monkeys received 10 trials per arm in each test session, with the arm being tested alternated for each trial. Each monkey was tested by the same investigator at the same time of day, 3 days per week, throughout the course of the study. The scientist testing the monkeys was blinded to the treatment group.

In the Global Primate Dyskinesia Rating Scale (“GPDRS”), each monkey was observed for the presence of dyskinesia one day per week prior to and following oral dosing using a Global Non-Human Primate Dyskinesia Rating Scale. Location of dyskinesias were noted. A validated dyskinesia scale was used once per week to determine the extent to which monkeys in each group develop dyskinesia. The Global Primate Dyskinesia Rating Scale used for this study was a single item scale with a 0 to 4 point range assessing the severity of dyskinesia with the scoring system shown in Table 1.

TABLE 1
GPDRS scoring

| Score | Observation |
|---|---|
| 0 | No evidence of dyskinesia |
| 1 | Subtle movements suggestive of dyskinesia, but could be normal |
| 2 | Mild dyskinesia: definitely present, but mild and of low amplitude |
| 3 | Moderate dyskinesia: intermediate or higher amplitude but not |
| | violent or extreme |
| 4 | Severe dyskinesia: extreme movements including flinging of the |
| | arms and/or violent jerks or thrust of the extremities or trunk; |
| | may have marked gyrations of the hips (hoola-hooping); may |
| | be incapacitating |

In the Skin Irritation Assessment, the site of the NP201 implant was assessed for presence or absence of irritation weekly at the same time as the blood draws were completed. Local irritation at the site of implantation was scored according to the Skin Irritation Score in Table 2.

TABLE 2
Skin Irritation Assessment scoring

| Score | Observation |
|---|---|
| 0 | No redness; |
| 1 | Minimal redness |
| 2 | Moderate redness with sharply defined borders |
| 3 | Intense redness without swelling |
| 4 | Intense redness with swelling |
| 5 | Intense redness with blistering/erosion |

All implantation of rods was done under sterile conditions. Implant size was to approximately 2 mm wide by 2 cm long. The monkeys received 3 mg/kg ketamine and 0.3 mg/kg of dormotor for anesthesia. The monkeys were shaved between the scapulas; the skin was then washed with betadine solution and alcohol. Implants were inserted either using a 10-12 gauge trochar or scalpel to create an approximate 2 cm incision. Implants were then placed under the skin and skin was closed with 3.0 vicrol suture(s).

The primary efficacy endpoints were reduction in Clinical Rating Scale score, improvement in Fine Motor Pick-up Test score and reversal of MPTP induced disruption in activity using the AMS. The secondary efficacy endpoints included reduction in dyskinesia relative to oral ropinirole at 6-8 weeks.

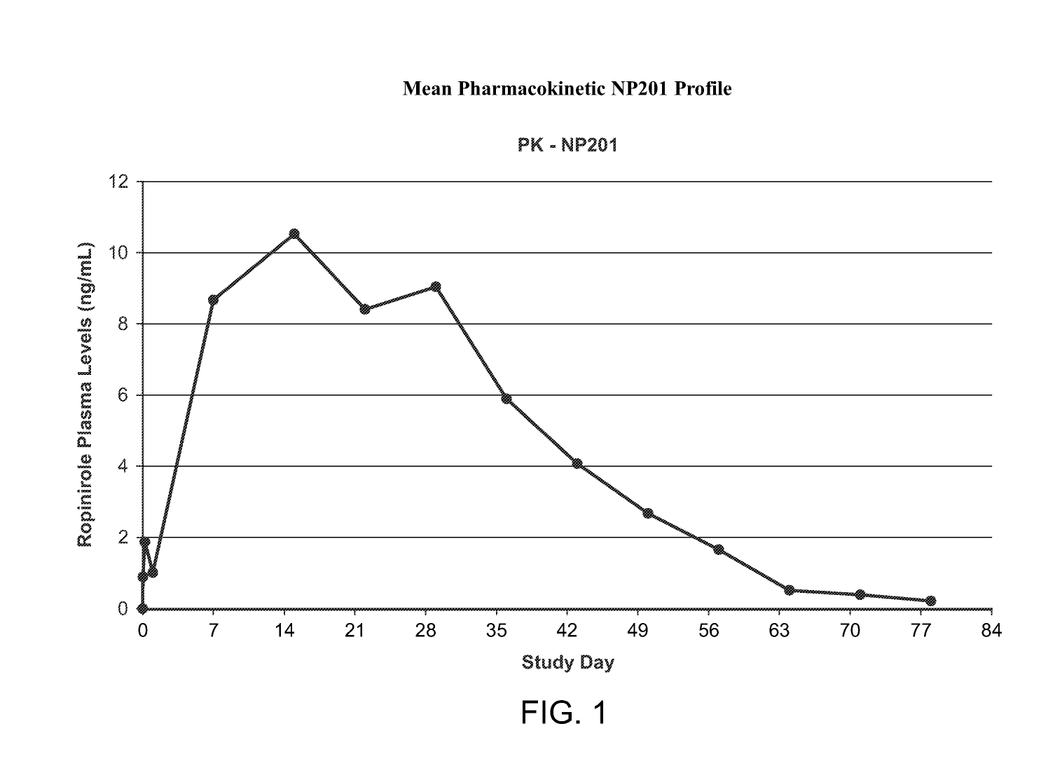

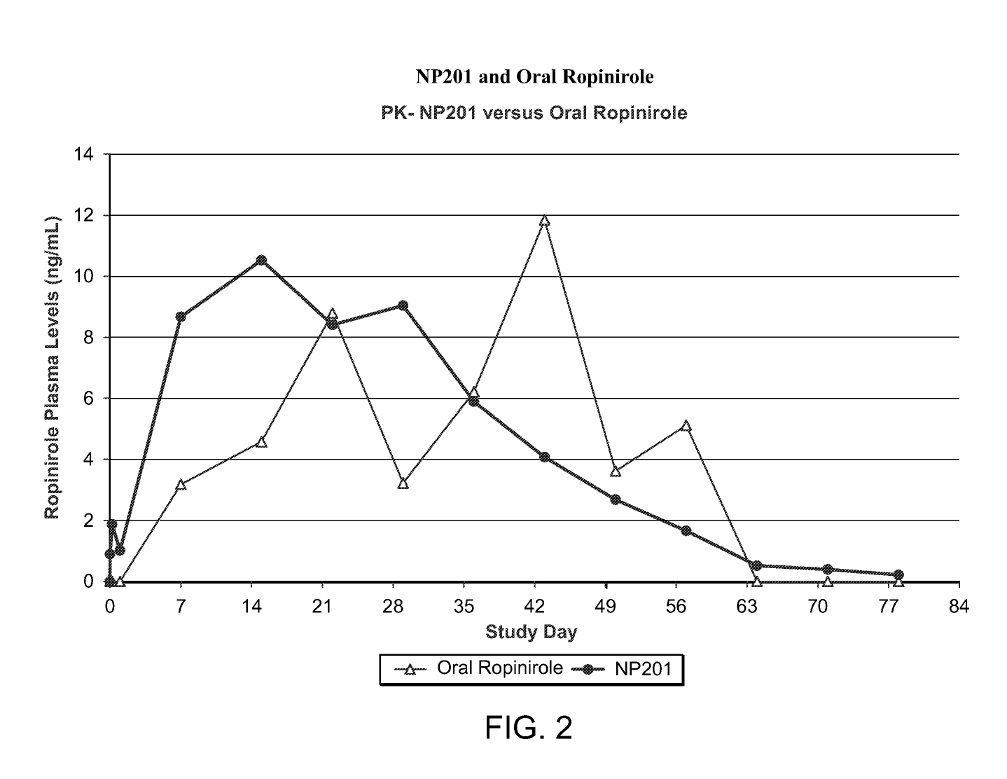

NP201 delivered detectable levels of ropinirole for 80 days, (see FIG. 1), with levels above 1 ng/ml between day 0 and day 58. NP201 serum levels remained between 4 and 11 ng/ml from day 7 to day 44 with steady decline thereafter. Oral ropinirole yielded levels between 3 and 8 ng/ml for the first 30 days, then increased to between 8 and 20 ng/ml for days 37 to 57 (see FIG. 2).

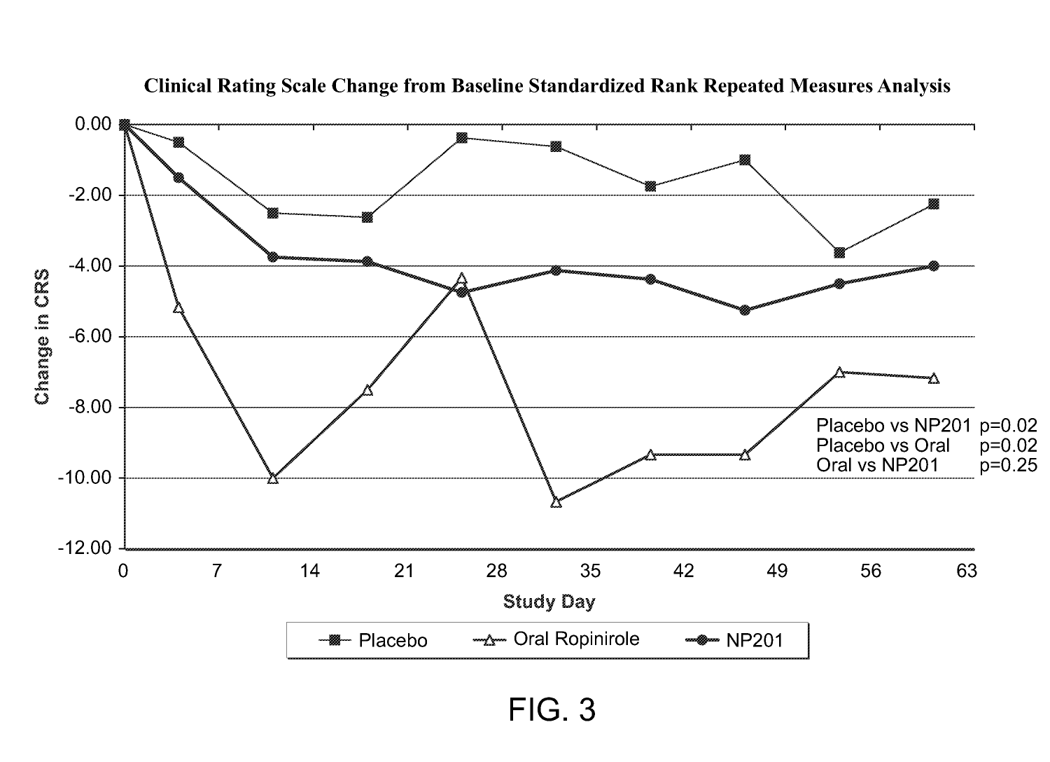

The Clinical Rating Scale change from baseline (see FIG. 3) showed Oral ropinirole was superior to placebo between 4 and 60 days. NP201 was superior to placebo between 11 and 46 days.

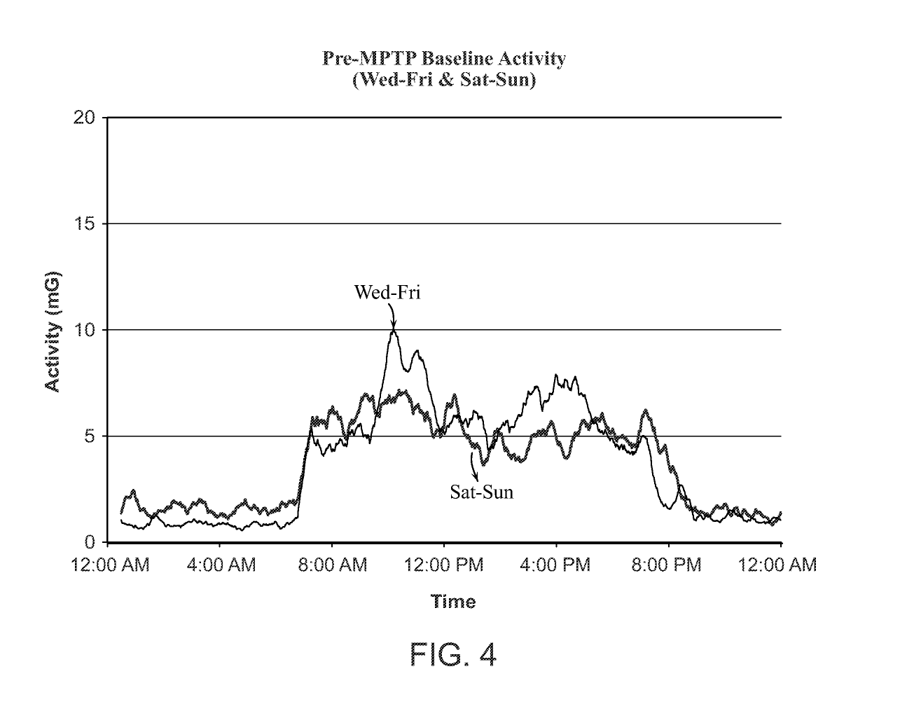

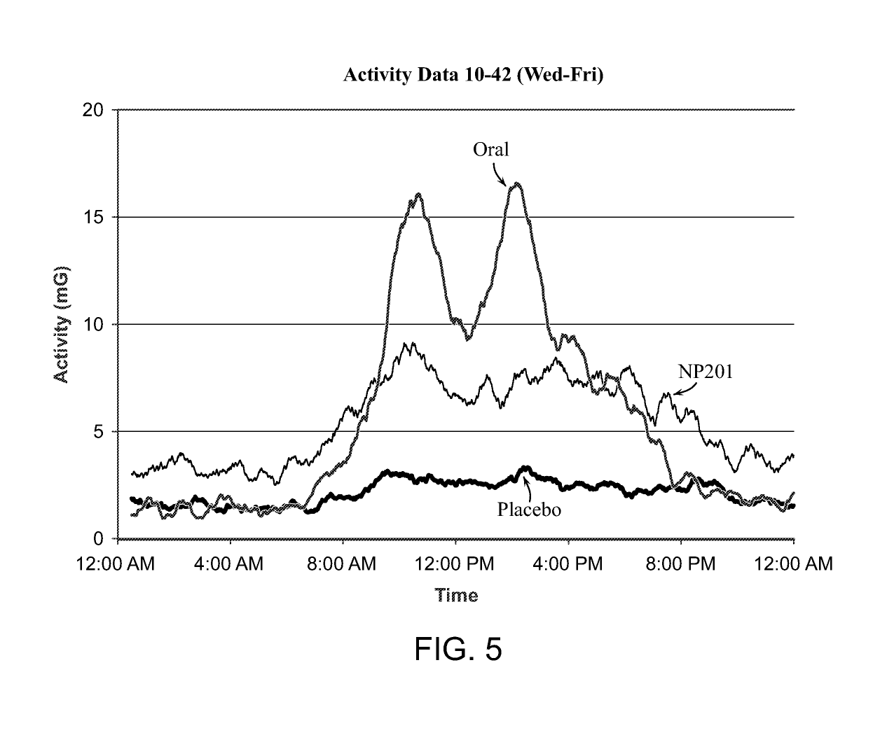

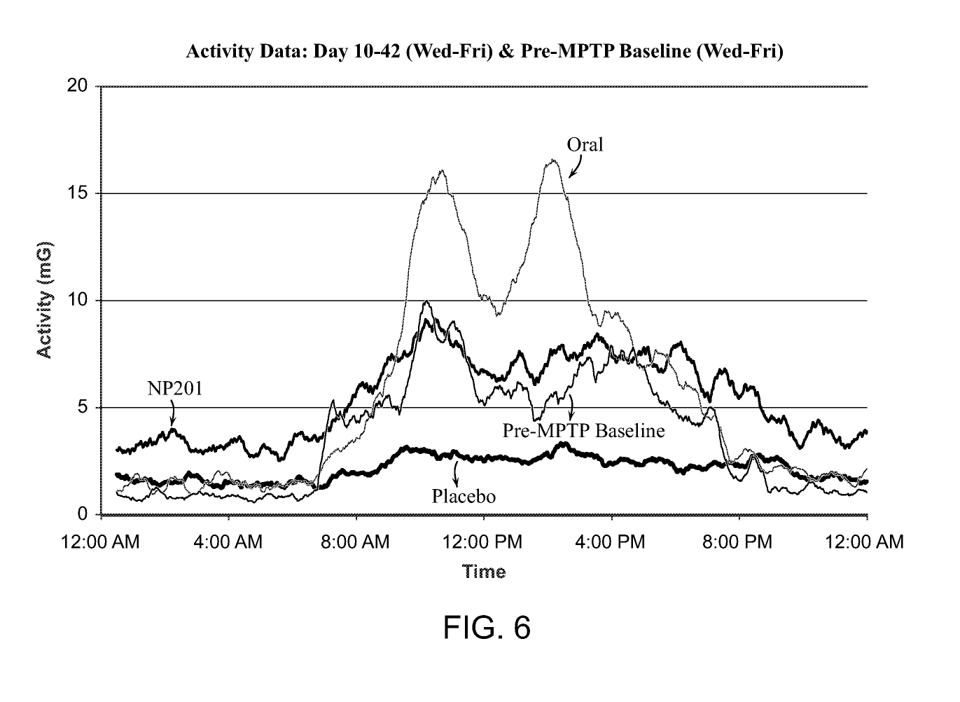

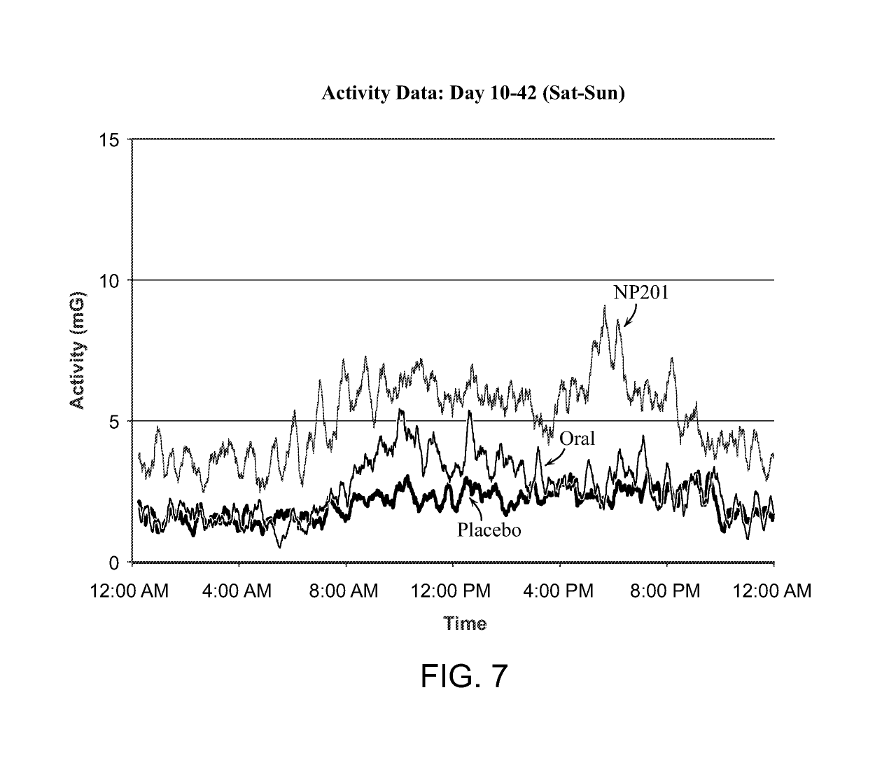

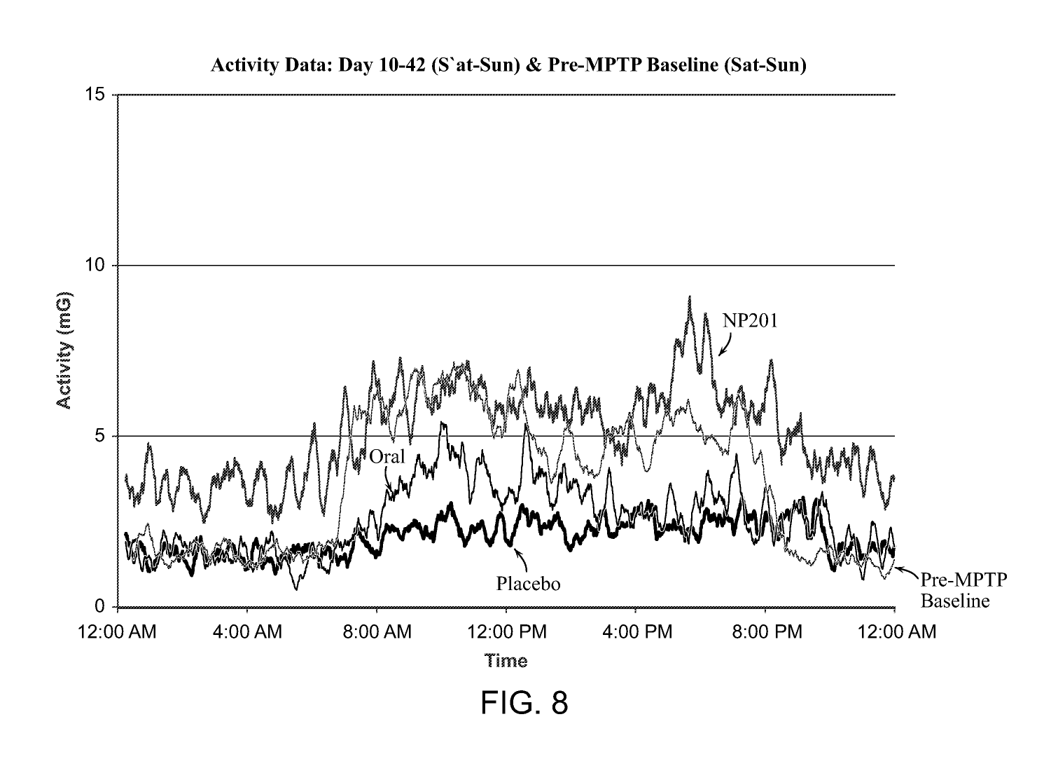

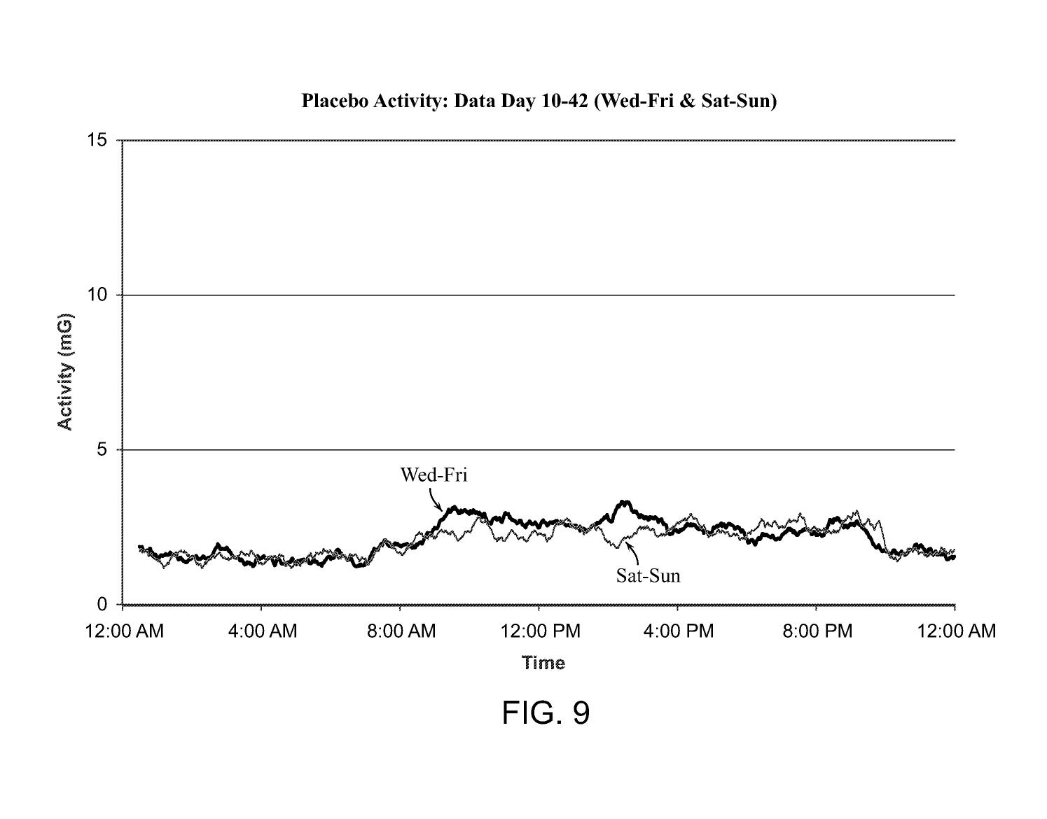

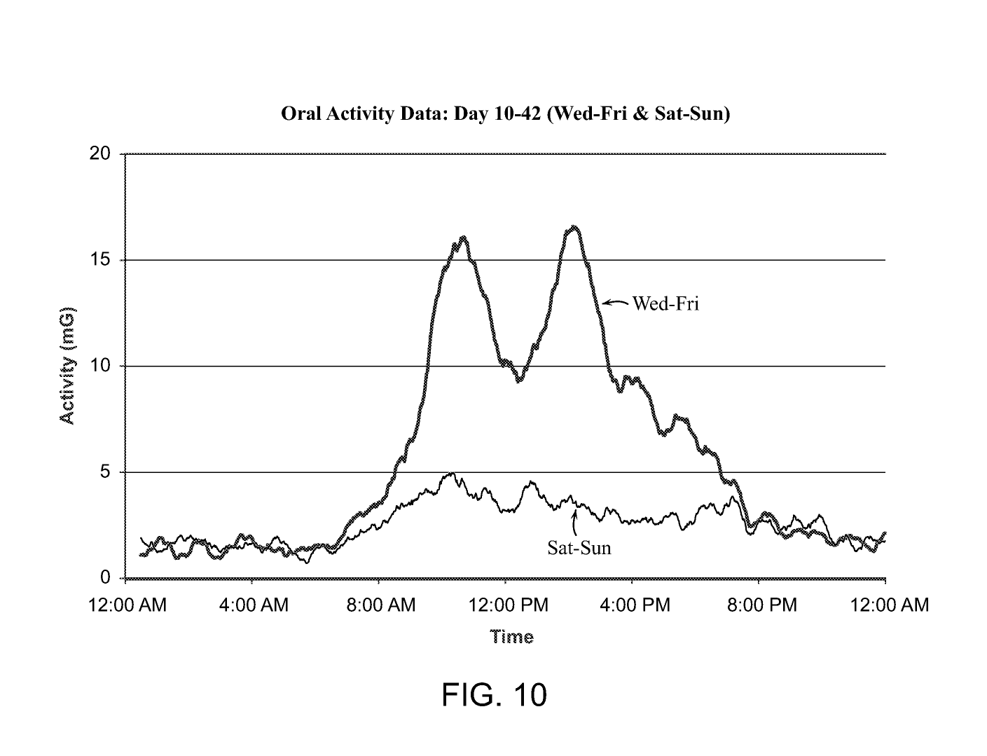

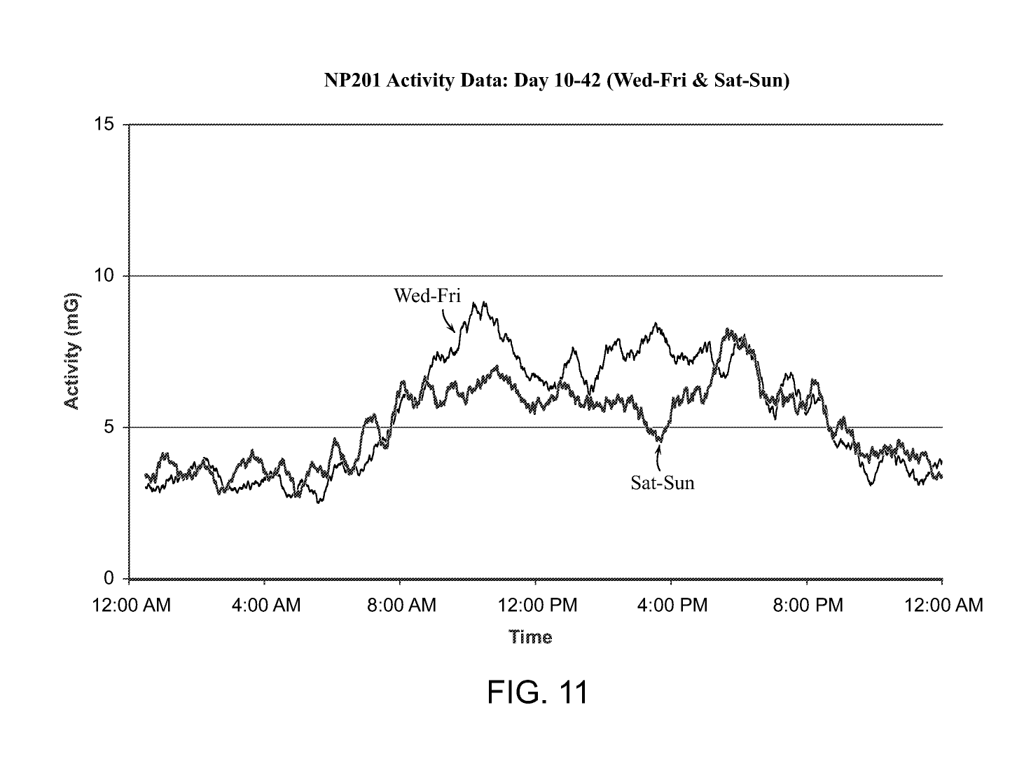

The activity can be summarized as follows: During Wed-Fri, oral ropinirole to yielded peak (see FIG. 5) activity levels at approximately 1 hour following each dose during the wake cycle that exceeded baseline, pre-MPTP levels (see FIG. 4). During the weekends, (Sat-Sun) the oral group resembled placebo with reduced activity relative to baseline (see FIG. 6, FIG. 7, FIG. 8, FIG. 9 and FIG. 10). NP201 restored normal levels and pattern of activity that was identical to pre-MPTP baseline levels for both the Wed-Fri and Sat-Sun periods (see FIG. 11).

Ropinirole implants achieve comparable efficacy to oral ropinirole for relief from motor impairments. Both oral ropinirole and NP201, at 1/9th of the oral dose, showed a CRS measure that was statistically superior to placebo (p<0.05). There was no significant difference (p>0.05) between NP201 and oral ropinirole for the period of time during which serum levels were comparable. This corresponded to 11-53 days post implantation.

A graphical representation of the data collected by the AMS (12-hour light/dark cycles) depicts the monkeys overall activity. Monkeys in the control group are continually bradykinetic while the oral group monkeys present cycles of activity while oral ropinirole is systemically available followed by periods of bradykinesia when it is not. The ropinirole implant groups present a 12-hour light/dark cycle similar to healthy non Parkinsonian monkeys. Although measurements of activity (mG) were slightly higher than normal during the dark cycles, there was no evidence that the sleep of the monkeys treated with NP201 was disturbed in any way. That is, activity data suggest that NP201 restored a pattern of normal, pre-MPTP levels of activity, while oral ropinirole yielded alternating periods of very high activity interspersed with normal levels. Furthermore, oral ropinirole treated animals were indistinguishable from placebo on weekend days, when they did not receive active agent, while NP201 animals displayed continuously normal activity throughout the active phase of the study.

There was much greater variability for CRS scores, activity and plasma levels in the oral ropinirole group relative to NP201. There were no signs of irritation at the site of implantation following either NP201 or placebo implants made of polymer alone.

In sum, NP201 delivered ropinirole for 80 days with clinically applicable levels for approximately 2 months. NP201 was superior to placebo on CRS, despite being administered at 1/9^{th} or 1/18^{th} of oral doses. Hyperactivity from oral ropinirole is consistent with animal models of stimulant induced psychosis, and with the observation to of medication induced psychotic episodes in PD patients on clinically appropriate doses of dopamine agonists. NP201 recreated the pattern and level of activity seen in the pre-MPTP baseline period, while oral ropinirole yielded alternating periods of very high and normal activity. Since dopamine agonist-induced hyperactivity in animals is predictive of psychotic effects in humans, low dose NP201 has the potential to provide clinical improvement in bradykinesia with less “off” periods and lower risk for medication induced psychosis. This data suggests that NP201 restored normal patterns of activity and improvement in clinical rating scores without hyperactivity.

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

1. A method for restoring normal patterns of activity in a subject suffering from Parkinson's Disease comprising:
administering an effective steady state concentration of a dopamine modulating compound continuously for a prolonged period of time such that normal patterns of activity are substantially restored in the subject.

2. A method for increasing on time in a subject suffering from Parkinson's Disease to comprising:
administering an effective steady state concentration of a dopamine modulating compound, alone or combination with another therapy, continuously for a prolonged period of time, such that on time is increased in the subject.

3. The method according to any one of the preceding claims, wherein off time is reduced in the subject.

4. The method according to any one of the preceding claims, wherein the severity of off time symptoms are reduced in the subject.

5. The method according to any one of the preceding claims, wherein the frequency of off time symptoms are reduced in the subject.

6. The method according to any one of the preceding claims, wherein the incidence of motor response complications are reduced in the subject.

7. The method according to any one of the preceding claims, wherein there are no significant and/or prolonged periods of hyperkinetic activity in the subject.

8. The method according to any one of the preceding claims, wherein there are no periods of hyperkinetic activity in the subject.

9. The method according to any one of the preceding claims, wherein the dopamine modulating compound is administered without the side effects associated with administration by pump infusion.

10. The method according to any one of the preceding claims, wherein the subject experiences a period of on time immediately after waking up from sleep.

11. The method according to any one of the preceding claims, wherein sustained efficacy is achieved in the subject for greater than 30 days.

12. The method according to any one of the preceding claims, wherein the delivery dose required to achieve the same pharmacokinetic profile as an approved orally administered dose is 1/9 or 1/18 that of the approved orally administered dose.

13. The method according to any one of the preceding claims, wherein the subject does not experience paralysis.

14. The method according to any one of the preceding claims, wherein the dopamine modulating compound is delivered via an implant.

15. The method according to any one of the preceding claims, wherein the dopamine modulating compound is delivered via an implant which comprises:
a core comprising a dopamine modulating compound and a first biodegradable polymer; and
a sheath comprising a second biodegradable polymer.

16. The method according to any one of the preceding claims, wherein the dopamine modulating compound is delivered via a depot.

17. The method according to any one of the preceding claims, wherein the dopamine modulating compound is co-administered with another therapy selected from dopamine metabolic inhibitors, monoamine oxidase inhibitors, dopaminergics, dopamine agonists or adenosine receptor antagonists.

18. The method according to claim 17, wherein the amount of the co-administered therapy administered is significantly decreased over time.

19. The method according to claim 18, wherein the side effects corresponding to the co-administered therapy are significantly reduced.

20. The method according to claim 19, wherein the co-administered therapy is a dopaminergic.

21. The method according to claim 20, wherein the dopaminergic is L-Dopa.

22. The method according to any one of the preceding claims, wherein the dopamine modulating compound is a 4-alkylamino-2(3H)-indolone compound.

23. The method according to any one of the preceding claims, wherein the dopamine modulating compound is selected from bromocriptine, pergolide, pramipexole, ropinirole, piribedil, cabergoline, and lisuride

24. The method according to any one of the preceding claims, wherein the dopamine modulating compound is ropinirole.

25. A method for the treatment of Parkinson's Disease in a patient in need thereof comprising, administering a continuous and prolonged delivery of ropinirole via an implant, in combination with L-Dopa, wherein on time is increased and off time is decreased.

26. The method according to any one of the preceding claims, wherein the subject is capable of normal activity during sleep.

27. The method according to any one of the preceding claims, wherein the patient is capable of normal movement continuously.

28. The method according to any one of the preceding claims, wherein the dopamine modulating compound is administered such that normal patterns of activity are substantially restored in the subject immediately after waking up from sleep.

29. The method according to any one of the preceding claims, wherein the dopamine modulating compound is administered such that normal patterns of activity are substantially restored in the subject for at least 18 hours per day.

30. The method according to any one of the preceding claims, wherein the Parkinson's Disease is mild to moderate Parkinson's Disease.
